(19) United States
(12) Patent Application Publication
(10) Pub. No.: US 2010/0070049 A1
(43) Pub. Date: Mar. 18, 2010

(54) METHOD AND APPARATUS FOR TREATING COMPRESSION FRACTURES IN VERTEBRAL BODIES

(76) Inventors: Patrick O'Donnell, Weston, MA (US); Hari Sundram, Brookline, MA (US); Mark Johanson, Littleton, MA (US)

Correspondence Address: Mark J. Pandiscio;Pandiscio & Pandiscio, P.C., 470 Totten Pond Road, Waltham, MA, 02451, US

(21) Appl. No.: 12/436,528

(22) Filed: May 6, 2009

Related U.S. Application Data
(60) Provisional application No. 61/126,684, filed on May 6, 2008.
(60) Provisional application No. 61/201,026, filed on Dec. 5, 2008.

(51) Int. Cl. A61F 2/28; A61B 17/56

(52) U.S. Cl. 623/23.56; 606/86 R

(57) ABSTRACT

A composite vertebroplasty cement for positioning in a void in a vertebral body so as to treat a compression fracture in that vertebral body, the composite vertebroplasty cement comprising:
a ceramic component for integrating with the vertebral body so as to treat the compression fracture; and
a sealing component for sealing the void in the vertebral body so as to maintain the composite vertebroplasty cement within the void in the vertebral body.

A composite vertebroplasty cement for positioning in a void in a vertebral body so as to treat a compression fracture in that vertebral body, the composite vertebroplasty cement comprising:
a conventional PMMA bone cement component for integrating with the vertebral body so as to treat the compression fracture; and
a sealing component for sealing the void in the vertebral body so as to maintain the composite vertebroplasty cement within the void in the vertebral body.

### REFERENCE TO PENDING PRIOR PATENT APPLICATIONS

This patent application claims benefit of:

(i) pending prior U.S. Provisional Patent Application Ser. No. 61/126,684, filed May 6, 2008 by Patrick O'Donnell et al. for NOVEL VERTEBROPLASTY CEMENT, AND NOVEL CEMENT CONTAINMENT SYSTEM, FOR USE IN TREATING COMPRESSION FRACTURES (Attorney's Docket No. MEDCAP-2 PROV); and

(ii) pending prior U.S. Provisional Patent Application Ser. No. 61/201,026, filed Dec. 5, 2008 by Patrick O'Donnell et al. for NOVEL VERTEBROPLASTY CEMENT, AND NOVEL CEMENT CONTAINMENT SYSTEM, FOR USE IN TREATING COMPRESSION FRACTURES (Attorney's Docket No. MEDCAP-4 PROV).

The two above-identified patent applications are hereby incorporated herein by reference.

### FIELD OF THE INVENTION

This invention relates to orthopedic surgery in general, and more particularly to methods and apparatus for treating compression fractures in vertebral bodies.

### BACKGROUND OF THE INVENTION

Compression fractures in vertebral bodies are a common occurrence, particularly among the elderly and the physically active (e.g., young athletes). Compression fractures can lead to serious deterioration of the spine and can cause substantial pain to the patient.

One current treatment for compression fractures involves injecting bone cement (i.e., polymethylmethacrylate or “PMMA”) into the interior of the vertebral body so as to stabilize the fracture and relieve the pain. More particularly, in this procedure, an opening is first made into the interior of the vertebral body, then a cavity is created in the interior of the vertebral body, and finally the bone cement (i.e., the PMMA) is injected into the cavity. The bone cement then hardens and provides relief to the patient.

The foregoing procedure is sometimes referred to as a vertebroplasty procedure, and the apparatus used to accomplish the same is sometimes referred to as a vertebroplasty cement system.

Kyphon Inc. of Sunnyvale, Calif., among others, has developed substantial technology in the area of vertebroplasty procedures and vertebroplasty cement systems.

Unfortunately, current vertebroplasty cement systems all suffer from a significant drawback, namely, that if the PMMA (i.e., the bone cement) extravasates out of the vertebral cavity, it can cause significant harm to the patient. By way of example but not limitation, if the PMMA extravasates out of the vertebral cavity and enters the blood stream, it can create a blood clot and result in a dangerous embolism. Furthermore, if the PMMA extravasates out of the vertebral cavity and encounters neural tissue, it can create neural necrosis (e.g., due to the PMMA's substantial exothermic properties). In this respect it will be appreciated that the PMMA is relatively viscous and must generally be injected into the bone cavity under pressure, so there is fair chance that the PMMA can migrate from the injection site to locations outside the vertebral body, e.g., through fractures in the vertebral body.

### SUMMARY OF THE INVENTION

The present invention is intended to address the foregoing problems by providing a new method and apparatus for treating compression fractures in vertebral bodies. More particularly, the present invention provides a new method and apparatus for performing a vertebroplasty procedure which substantially eliminates the risk of bone cement migration out of the vertebral body.

In one form of the invention, there is provided a composite vertebroplasty cement for positioning in a void in a vertebral body so as to treat a compression fracture in that vertebral body, the composite vertebroplasty cement comprising:

a ceramic component for integrating with the vertebral body so as to treat the compression fracture; and

a sealing component for sealing the void in the vertebral body so as to maintain the composite vertebroplasty cement within the void in the vertebral body.

In another form of the invention, there is provided a method for treating a compression fracture in a vertebral body, the method comprising the steps of:

providing a composite vertebroplasty cement for positioning in a void in a vertebral body so as to treat a compression fracture in that vertebral body, the composite vertebroplasty cement comprising:
a ceramic component for integrating with the vertebral body so as to treat the compression fracture; and
a sealing component for sealing the void in the vertebral body so as to maintain the composite vertebroplasty cement within the void in the vertebral body; and

positioning the composite vertebroplasty cement in the void in the vertebral body so as to treat the compression fracture in that vertebral body.

In another form of the invention, there is provided a composite vertebroplasty cement for positioning in a void in a vertebral body so as to treat a compression fracture in that vertebral body, the composite vertebroplasty cement comprising:

a conventional PMMA bone cement component for integrating with the vertebral body so as to treat the compression fracture; and

a sealing component for sealing the void in the vertebral body so as to maintain the composite vertebroplasty cement within the void in the vertebral body.

In another form of the invention, there is provided a method for treating a compression fracture in a vertebral body, the method comprising the steps of:

providing a composite vertebroplasty cement for positioning in a void in a vertebral body so as to treat a compression fracture in that vertebral body, the composite vertebroplasty cement comprising:
a conventional PMMA bone cement component for integrating with the vertebral body so as to treat the compression fracture; and
a sealing component for sealing the void in the vertebral body so as to maintain the composite vertebroplasty cement within the void in the vertebral body; and

positioning the composite vertebroplasty cement in a void in the vertebral body so as to treat a compression fracture in that vertebral body.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIG. 1 is a schematic view showing a novel vertebroplasty cement formed in accordance with the present invention;

FIG. 2 is a schematic view showing the vertebroplasty cement of FIG. 1 being injected into a vertebral body;

FIG. 3 is a schematic view showing another novel vertebroplasty cement formed in accordance with the present invention;

FIG. 4 is a schematic view showing the vertebroplasty cement of FIG. 3 being injected into a vertebral body;

FIG. 5 is a schematic view showing another novel vertebroplasty cement formed in accordance with the present invention;

FIGS. 6 and 7 are schematic views showing the vertebroplasty cement of FIG. 5 being injected into a vertebral body;

FIG. 8 is a schematic view showing alternative apparatus for injecting the vertebroplasty cement of FIG. 5 into a vertebral body;

FIG. 9 is a schematic view showing another novel vertebroplasty cement formed in accordance with the present invention;

FIGS. 10 and 11 are schematic views showing the vertebroplasty cement of FIG. 9 being injected into a vertebral body; and

FIG. 12 is a schematic view showing alternative apparatus for injecting the vertebroplasty cement of FIG. 9 into a vertebral body

### DETAILED DESCRIPTION OF THE PREFERRED EMBODIMENTS

The present invention provides a new method and apparatus for treating compression fractures in vertebral bodies. More particularly, the present invention provides a new method and apparatus for performing a vertebroplasty procedure which substantially eliminates the risk of bone cement migration out of the vertebral body.

### Novel Composite Vertebroplasty Cement Comprising Ceramic Component Coated with Sealing Component

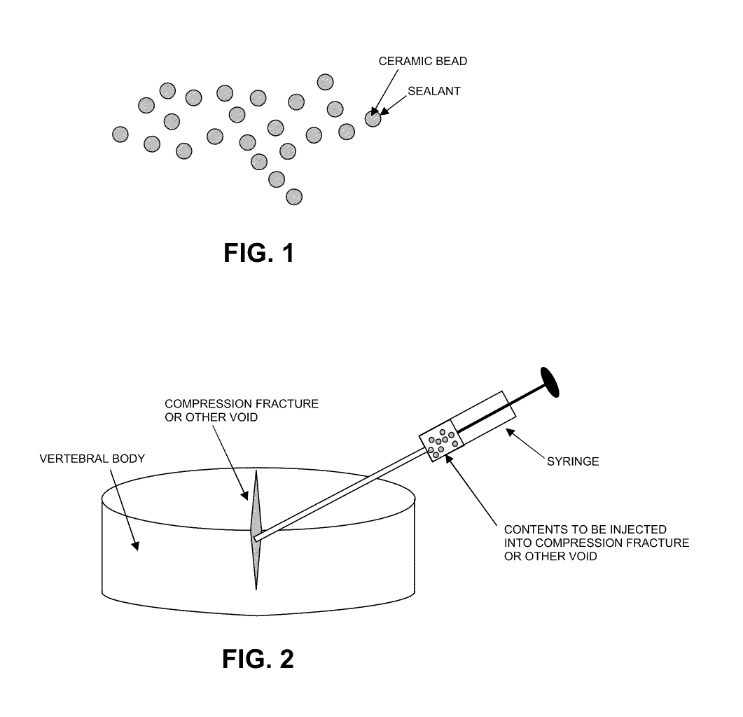

In one form of the present invention, there is provided a novel composite vertebroplasty cement which comprises two parts: (i) a ceramic component (e.g., hydroxyapatite, tricalcium phosphate, calcium aluminate, etc.), and (ii) a polymer or fibrin sealing component (e.g., polyethylene glycol or “PEG”, carboxymethylcellulose or “CMC”, fibrin, polyvinylalcohol or “PVA”, etc.). The sealing component is coated on the outside of the ceramic particles. See FIG. 1. Thus, the sealing component is effectively bundled with the ceramic component so that the two can be delivered to the fractured interior of the vertebral body in conjunction with one another.

In use, a cavity may or may not be created in the vertebral body in the traditional manner, and then the novel composite vertebroplasty cement is injected into the vertebral fracture. See FIG. 2. When blood in the vertebral body reacts with the sealing component, the blood activates the sealing component, causing the sealing component to quickly adhere and set, thereby locking the vertebroplasty cement within the vertebral body. More particularly, when blood in the vertebral body encounters the vertebroplasty cement in the cavity, the cement's sealing component activates so as to adhere and set. This action converts the heretofore-fluid vertebroplasty cement into a solid mass which adheres to the walls of the cavity. Thus, the vertebroplasty cement is sealed within the cavity in the vertebral body, with the ceramic component present to address the compression fracture.

Thus, the novel composite vertebroplasty cement (i) is an easily flowable mixture prior to exposure to blood in the vertebral body, whereby it can be injected into the cavity in the vertebral body, (ii) comprises a ceramic material which can integrate into the vertebral body so as to address the compression fracture, and (iii) comprises a sealing component which, when contacted by blood in the vertebral body, quickly adheres and sets, thereby locking the composite vertebroplasty cement in position within the vertebral body and eliminating the danger of cement extravasation. Significantly, since the novel composite vertebroplasty cement contains no PMMA, little harm will be done to the body even if some cement extravasation should inadvertently occur.

### Composite Vertebroplasty Cement Comprising a Slurry of the Ceramic Component and Sealing Component

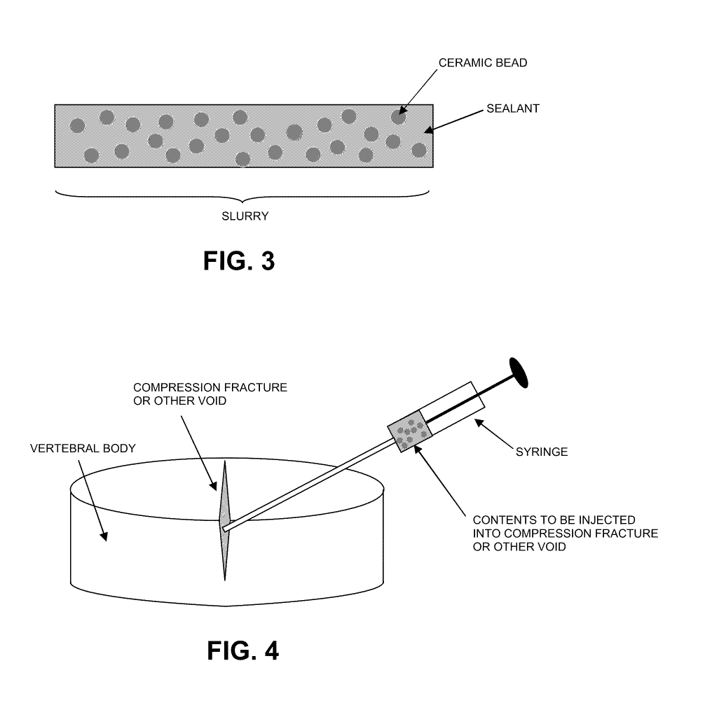

In another form of the present invention, there is provided a novel composite vertebroplasty cement which comprises a slurry of the aforementioned ceramic component and the aforementioned sealing component. See FIG. 3. Thus, in this form of the invention, the sealing component is again effectively bundled with the ceramic component so that the two can be delivered to the fractured interior of the vertebral body in conjunction with one another.

In use, a cavity may or may not be created in the vertebral body in the traditional manner, and then the novel composite vertebroplasty cement is injected into the vertebral fracture. See FIG. 4. When blood in the vertebral body reacts with the sealing component, the blood activates the sealing component, causing the sealing component to quickly adhere and set, thereby locking the vertebroplasty cement within the vertebral body. More particularly, when blood in the vertebral body encounters the vertebroplasty cement in the cavity, the cement's sealing component activates so as to adhere and set. This action converts the heretofore-fluid vertebroplasty cement into a solid mass which adheres to the walls of the cavity. Thus, the vertebroplasty cement is sealed within the cavity in the vertebral body, with the ceramic component present to address the compression fracture.

Thus, the novel composite vertebroplasty cement (i) is an easily flowable mixture prior to exposure to blood in the vertebral body, whereby it can be injected into the cavity in the vertebral body, (ii) comprises a ceramic material which can integrate into the vertebral body so as to address the compression fracture, and (iii) comprises a sealing component which, when contacted by blood in the vertebral body, quickly adheres and sets, thereby locking the composite vertebroplasty cement in position within the vertebral body and eliminating the danger of cement extravasation. Significantly, since the novel composite vertebroplasty cement contains no PMMA, little harm will be done to the body even if some cement extravasation should inadvertently occur.

### Serial Delivery of the Sealing Component and the Ceramic Component of the Composite Vertebral Cement

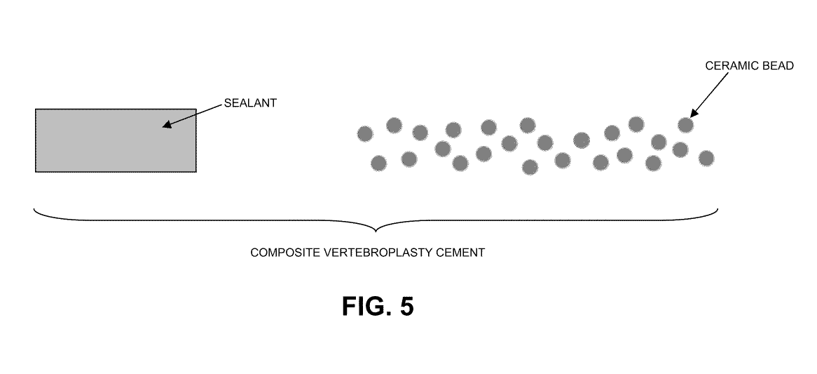

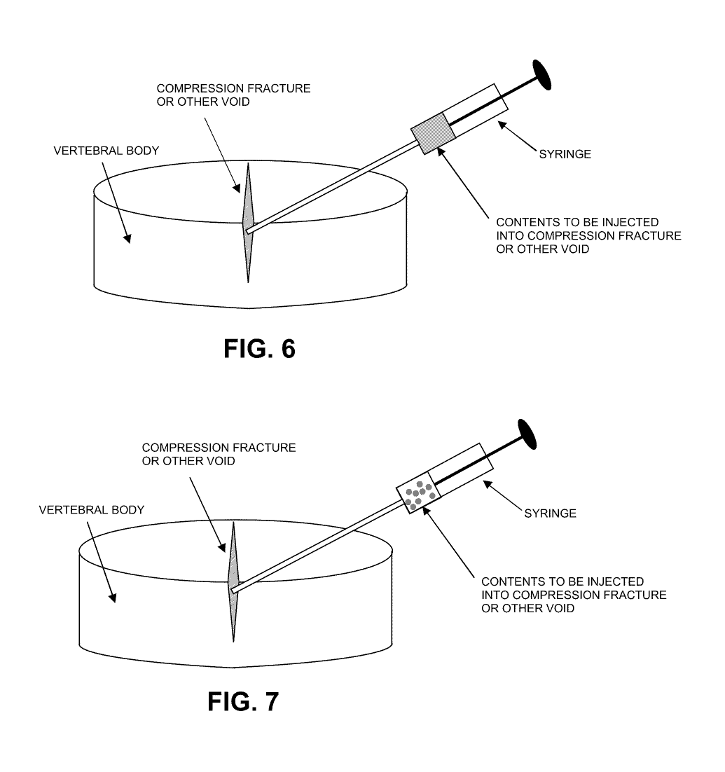

In another form of the present invention, the composite vertebroplasty cement can comprise two separate components delivered in a serial fashion. More particularly, in this form of the invention, the composite vertebroplasty cement comprises the aforementioned ceramic component and the aforementioned sealing component. See FIG. 5. However, the components are kept segregated from one another prior to use. Then, at the time of use, the sealing component is delivered first so as to fill in the intersticies in the fractured vertebra. See FIG. 6. When blood in the vertebral body encounters the vertebroplasty cement in the cavity, the sealing component activates so as to set and thereby seal the intersticies of the fractured vertebra at the peripheries of the intersticies, i.e., at the locations where blood can contact the sealing component. Thereafter, the ceramic component is injected into the bone void which has been sealed with the sealing component. See FIG. 7. The ceramic component is thereafter locked to the sealing component as more blood seeps into the sealing component. If desired, more sealing component can thereafter be added to ceramic component so as to further seal off the mass. As a result, the composite cement can address the fracture in the vertebra while still eliminating cement extravasation out of the vertebral body.

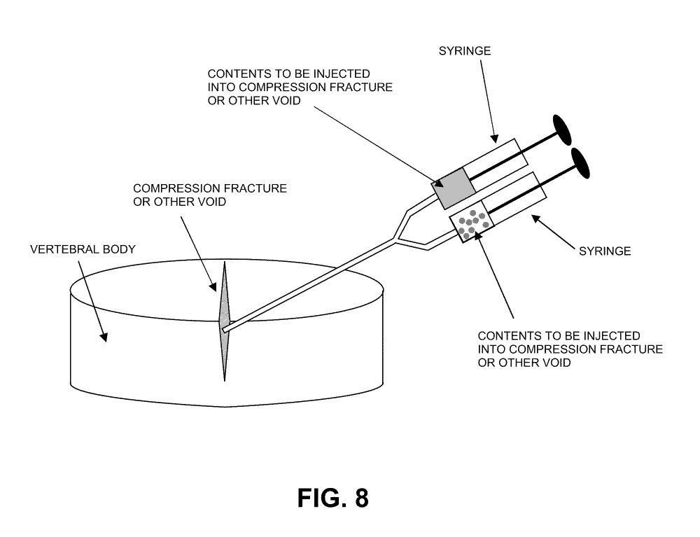

If desired, the sealing component and the ceramic component can be packaged into a dual-chamber syringe so that the syringe needle does not need to be removed and re-inserted between component deployments. See FIG. 8.

### Composite Vertebroplasty Cement Comprising a Sealing Component and Conventional PMMA Bone Cement, with the Sealing Component and the Conventional PMMA Bone Cement being Delivered Serially

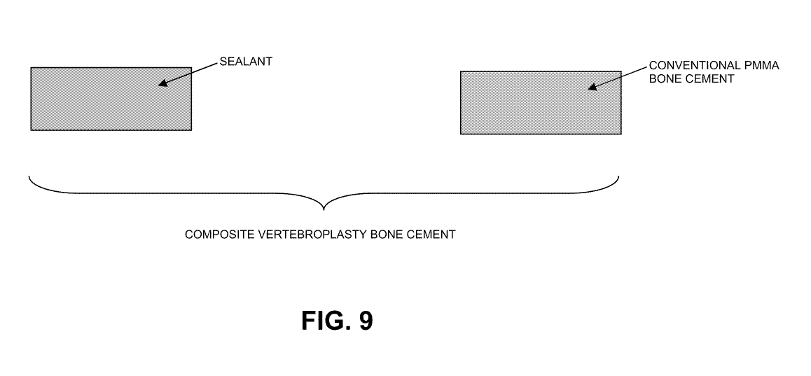

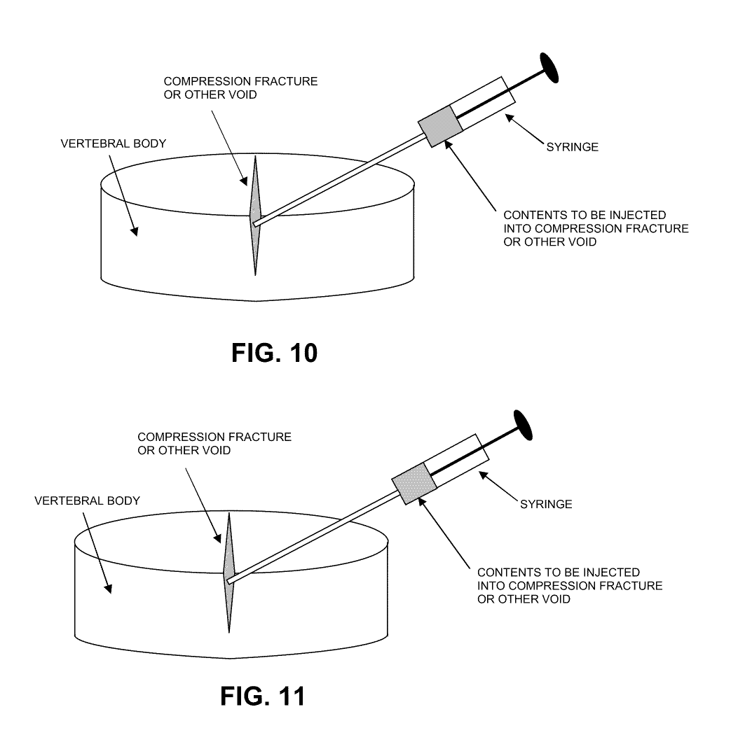

In another form of the present invention, there is provided a composite vertebroplasty cement which comprises a sealing component and conventional PMMA bone cement. More particularly, in this form of the invention, the sealing component may comprise the polymer or fibrin sealing component (e.g., polyethylene glycol PEG, carboxymethylcellulose, fibrin, polyvinylalcohol PVA, etc.) discussed previously. The conventional bone cement can comprise polymethylmethacrylate (PMMA) bone cement. See FIG. 9. The sealing component is delivered first, so as to fill in and seal the intersticies in the fractured vertebra. See FIG. 10. Then the conventional bone cement (e.g., polymethymethylacrylate PMMA, or ceramic material such as TCP/HA etc.) can be safely injected into the opening, with the sealing component retaining the bone cement within the bone and preventing leakage therefrom. See FIG. 11. Preferably, the conventional bone cement is then “capped” with a further layer of sealing component, with the capping layer of sealing component thereafter adhering and setting, whereby to completely seal in the PMMA bone cement within the bone. The PMMA bone cement can thereafter cure in situ so as to provide the desired structural integrity to the bone, without fear of cement extravasation.

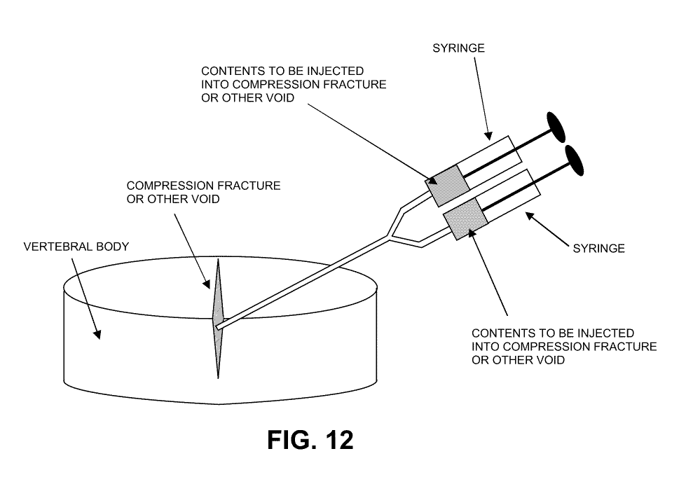

If desired, the sealing component and the conventional PMMA bone cement can be packaged into a dual-chamber syringe so that the syringe needle does not need to be removed and then re-inserted between component deployments. See FIG. 12.

In one preferred form of the invention, the sealing component is engineered and configured so that:

(i) the sealing component can reliably prevent conventional bone cement extravasation, whereby to minimize the possibility of embolisms, exothermal nerve root damage and/or hypotension—and by configuring the sealing component so that it can reliably prevent cement extravasation, the need to use cavity creation devices (e.g., inflatable balloons) to prevent cement extravasation can be eliminated (of course, it may still be desirable to use cavity creation devices for other purposes, e.g., for height restoration in a fractured vertebral body, etc.); and/or

(ii) the sealing component can serve as a “heat sink” for the exothermic reactions of the PMMA bone cement; and/or

(iii) the sealing component can serve as a cushion to modify the stiffness of the PMMA implant, whereby to minimize endplate fractures of the vertebral body; and/or

(iv) the sealing component can serve to encapsulate tricalciumphosphate-hydroxyapatite (TCP/HA) ceramic injectables to prevent giant cell infiltration.

Among other things, the sealing component may comprise a polymer.

Where the sealing component comprises a polymer, the polymer may be non-degradable or degradable. If the polymer is degradable, the polymer is configured so that the degradation products are non-toxic and preferably eliminated from the site of implantation. If the polymer is non-degradable, the polymer is engineered so that the long term stability of the solid polymer is satisfactory for vertebroplasty cement applications. Preferably the monomeric units of the polymer chain are of the sort well known to the FDA.

Furthermore, where the sealing component is a polymer, the polymer is preferably engineered and configured so that it has the ability to go through a phase change, e.g., from a flowable liquid at the time of injection to an elastic solid at the site of implantation in the body. This phase change can be either physical or chemical in nature. Furthermore, this phase change can be reversible or non-reversible in nature. By way of example but not limitation, the polymer may comprise a phase change elastic thermal plastic, or a phase change hydrogel, etc.

In one preferred form of the invention, the sealing component is engineered and configured so that it includes dimethylacrylamide (DMA). The DMA acts as a plasticizer for the polymer as well as to stimulate an osteoinductive bone regeneration cascade.

Preferably the polymer is stable at room temperature, does not employ the use of catalysts such as metals, and is sterilizable (e.g., via sterile filtration, gamma irradiation, etc).

Thus it will be seen that the novel method and apparatus of the present invention provides a significant advantage over prior art vertebroplasty methods and apparatus, successfully treating the compression fracture while reliably eliminating the risk of cement extravasation. More particularly, the method and apparatus of the present invention provides the desired therapeutic benefits needed to address compression fractures, while simultaneously substantially eliminating the risk of bone cement extravasation. As a result, the present invention effectively eliminates the clotting and embolism issues, and the neural necrosis issues, present with conventional vertebroplasty therapies.

It should be appreciated that the present invention may also be used for bone grafting applications. More particularly, the present invention can be used to temporarily adhere a bone graft to a host bone, with the novel composite bone cement providing both adherence and an osteoconductive matrix.

The present invention may also be used for numerous other bone therapies which will be apparent to those skilled in the art in view of the present disclosure.

### Modifications of the Preferred Embodiments

It should be understood that many additional changes in the details, operation, steps and arrangements of elements, which have been herein described and illustrated in order to explain the nature of the present invention, may be made by those skilled in the art while still remaining within the principles and scope of the invention.

What is claimed is:

1. A composite vertebroplasty cement for positioning in a void in a vertebral body so as to treat a compression fracture in that vertebral body, the composite vertebroplasty cement comprising:
a ceramic component for integrating with the vertebral body so as to treat the compression fracture; and
a sealing component for sealing the void in the vertebral body so as to maintain the composite vertebroplasty cement within the void in the vertebral body.

2. A composite vertebroplasty cement according to claim 1 wherein the ceramic component comprises a plurality of particles, and further wherein the sealing component comprises coatings on the plurality of particles.

3. A composite vertebroplasty cement according to claim 1 wherein the ceramic component comprises a plurality of particles and the sealing component comprises a flowable mass, and further wherein the vertebroplasty cement comprises a slurry of the ceramic component particles in the flowable mass of the sealing component.

4. A composite vertebroplasty cement according to claim 1 wherein the ceramic component and the sealing component are segregated from one another prior to use.

5. A composite vertebroplasty cement according to claim 1 wherein the ceramic component comprises at least one from the group consisting of:
hydroxyapatite, tricalcium phosphate and calcium aluminate.

6. A composite vertebroplasty cement according to claim 1 wherein the sealing component comprises at least one from the group consisting of a polymer and fibrin.

7. A composite vertebroplasty cement according to claim 1 wherein the sealing component comprises at least one from the group consisting of polyethylene glycol (PEG), carboxymethylcellulose (CMC), fibrin and polyvinylalcohol (PVA).

8. A method for treating a compression fracture in a vertebral body, the method comprising the steps of:
providing a composite vertebroplasty cement for positioning in a void in a vertebral body so as to treat a compression fracture in that vertebral body, the composite vertebroplasty cement comprising:
a ceramic component for integrating with the vertebral body so as to treat the compression fracture; and
a sealing component for sealing the void in the vertebral body so as to maintain the composite vertebroplasty cement within the void in the vertebral body; and
positioning the composite vertebroplasty cement in the void in the vertebral body so as to treat the compression fracture in that vertebral body.

9. A method according to claim 8 wherein the ceramic component and the sealing component are simultaneously positioned in the void in the vertebral body.

10. A method according to claim 8 wherein the ceramic component and the sealing component are serially positioned in the void in the vertebral body.

11. A method according to claim 10 wherein the sealing component is positioned in the void in the vertebral body before the ceramic component is positioned in the void in the vertebral body.

12. A composite vertebroplasty cement for positioning in a void in a vertebral body so as to treat a compression fracture in that vertebral body, the composite vertebroplasty cement comprising:
a conventional PMMA bone cement component for integrating with the vertebral body so as to treat the compression fracture; and
a sealing component for sealing the void in the vertebral body so as to maintain the composite vertebroplasty cement within the void in the vertebral body.

13. A composite vertebroplasty cement according to claim 12 wherein the ceramic component and the sealing component are segregated from one another prior to use.

14. A composite vertebroplasty cement according to claim 12 wherein the sealing component comprises at least one from the group consisting of a polymer and fibrin.

15. A composite vertebroplasty cement according to claim 12 wherein the sealing component comprises at least one from the group consisting of polyethylene glycol (PEG), carboxymethylcellulose (CMC), fibrin and polyvinylalcohol (PVA).

16. A method for treating a compression fracture in a vertebral body, the method comprising the steps of:
providing a composite vertebroplasty cement for positioning in a void in a vertebral body so as to treat a compression fracture in that vertebral body, the composite vertebroplasty cement comprising:
a conventional PMMA bone cement component for integrating with the vertebral body so as to treat the compression fracture; and
a sealing component for sealing the void in the vertebral body so as to maintain the composite vertebroplasty cement within the void in the vertebral body; and
positioning the composite vertebroplasty cement in a void in the vertebral body so as to treat a compression fracture in that vertebral body.

17. A method according to claim 16 wherein the conventional PMMA bone cement component and the sealing component are serially positioned in the void in the vertebral body.

18. A method according to claim 17 wherein the sealing component is positioned in the void in the vertebral body before the PMMA bone cement component is positioned in the void in the vertebral body.
